(19) United States
(12) Patent Application Publication
(10) Pub. No.: US 2009/0157153 A1
(43) Pub. Date: Jun. 18, 2009

(54) Skin cooling system

(76) Inventors: Sarah Anne Lemke, Appleton, WI (US); Lisa Ann Flugge-Berendes, Appleton, WI (US); Tammy J. Balzar, Oshkosh, WI (US); Scott W. Wenzel, Neenah, WI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 12/002,224

(22) Filed: Dec. 13, 2007

(51) Int. Cl. A61F 7/10

(52) U.S. Cl. 607/114

(57) ABSTRACT

A system for cooling the skin including a cooling composition and an article made at least in part from a phase change material. The article may contain the cooling composition, or be separate therefrom. Different article forms include a vessel, a token, a figurine, jewelry, a packet, among other forms. The phase change material may encapsulate to form a plurality of microcapsules, or may form a single mass.

The present invention relates to cooling system for the skin, in particular, a cooling formulation combined with an article embodying a phase change material.

Some consumer products are directed toward cooling a person when they have a fever, hot flash, or are simply feeling too warm because of physical exertion or ambient temperatures. Such products may include icilin or other neurosensory materials, alcohol or other evaporative materials, or phase change materials.

When a person desires cooling of the skin, such as when a hot flash occurs, the desire may be for immediate relief. Persons that can anticipate the need for immediate relief may carry a liquid cooling formulation for selective application to the skin. However, sometimes the application of the formulation may be delayed or not practical for all parts of the body, for example, the palm of the hand. The user may only be able hold the package of cooling formulation for a period of time before the cooling composition can be applied to the skin. As such, there remains a need for a cooling formulation system that can provide thermal relief from a hot flash or the like prior to application.

### SUMMARY OF THE INVENTION

The present invention is a cooling system which includes a human skin cooling composition having a neurosensory component, and an article made with a first phase change material. The human skin cooling composition and the article are secured together by a package to form a kit.

In another aspect of the present invention, the cooling system includes a human skin cooling composition having a neurosensory component, and a vessel for containing the cooling composition. The vessel is made with a plurality of microcapsules.

In yet another aspect of the present invention, there is a method of marketing a skin cooling system. The method steps include: providing a skin cooling composition, providing an article comprising a phase change material, and providing instructions on how to use the skin cooling composition with the article to provide a cooling sensation on human skin.

Additional features and advantages will be apparent from the detailed description which follows, taken into conjunction with the accompanying drawings, which together illustrate, by way of example, features of the invention.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended FIGS. in which:

FIG. 1 is a schematic view of the present invention showing very generally a quantity of a cooling composition and an article made with a phase change material;

FIG. 2 is a front perspective view of one embodiment of an article of the present invention in the form of a vessel with an applicator;

FIG. 3 is a cross-sectional view of another embodiment of an article of the present invention in the form of a coating;

FIG. 4 is a front perspective view of another embodiment of an article of the present invention in the form of a pendant;

FIG. 5 is a front perspective view of another embodiment of an article of the present invention in the form of a packet;

FIG. 6 is a front perspective view of another embodiment of an article of the present invention in the form of a token; and

FIG. 7 is a front perspective view of another embodiment of an article of the present invention in the form of a figurine.

FIG. 8 is a front perspective view of one embodiment of the present invention, showing one embodiment of how it may be marketed.

### DETAILED DESCRIPTION OF THE PRESENT INVENTION

### Overview

The present invention is a skin cooling system that includes a cooling composition and phase change article that absorbs heat upon direct skin contact therewith. The phase change article may function as a container for the cooling composition, or may be another article, separate from the container of cooling composition. The advantage presented by this system is that it can enhance the consumer's experience of using the cooling formulation, and provide an additional measure of cooling relief before, during, and after the application of a cooling formulation.

The cooling composition and one or more articles may be packaged together to form a kit. Thus, the cooling system may be secured together by packaging, including but not limited to a wrapper (e.g. cellophane or paper), a box, a crate, a can, a basket, or any other housing. In addition, packaging may be as simple as a string, tape, or glue used to secure the cooling composition and the article(s) together. Of course, as the cooling composition is a liquid, if not contained within an article, it will be contained in a container (e.g. a bottle, pouch, or can).

Reference will now be made in detail to embodiments of the invention, one or more examples of which are illustrated in the drawings. Each example is provided by way of explanation of the invention, and not meant as a limitation of the invention. For example, features illustrated or described as part of one embodiment can be used with another embodiment to yield still a third embodiment. It is intended that the present invention include these and other modifications and variations.

### DEFINITIONS

As used herein, the singular terms “a,” “an,” and “the” include plural referents unless the context clearly dictates otherwise. Thus, for example, reference to a phase change material can include multiple phase change materials unless the context clearly dictates otherwise.

As used herein, “cooling composition” in the context of the present invention is defined as a good that is capable of changing temperature or providing a sensation of changing temperature as detected by human skin or mucous membranes. The thermal consumer product may feel either cooler or warmer than ambient temperature. The thermal consumer product may undergo a phase change to impart an actual temperature change, or contain a neurosensory material for a sensation of temperature change. Cooling compositions may include those described in a patent application entitled SKIN COOLING COMPOSITION, U.S. patent Ser. No. 11/981,344, filed on Oct. 31, 2007, incorporated herein. Generally, cooling compositions may include one or more neurosensory components, one or more evaporative components, and/or one or more phase change components. For example, a cooling composition may contain one or more of the following ingredients: water, alcohol, menthol or icilin, fragrance, hydroxyethyl acrylate, sodium acryloyldimethyltaurate copolymer, squalane, lauryl laurate, ethyl menthane, carboxamides, methyl diisopropyl propionamide, menthyl lactate, hydroxyethyl behenamidoproyl dimoniumchloride, and polysorbate.

As used herein, the term “microcapsules” refers to shells that define internal compartments within which a phase change material is positioned. Microcapsules can be formed of any suitable material that serves to contain the phase change material, thus offering a degree of protection to the phase change material against ambient or processing conditions or against loss or leakage during use. For example, the microcapsules can be formed of a polymer or any other suitable encapsulation material. For certain implementations, the microcapsules can be formed of melamine-formaldehyde or urea-formaldehyde by in-situ polymerization. The selection of a material forming the microcapsules can be dependent upon other considerations, such as based on its affinity for a binder, its reactivity or lack of reactivity with the phase change material, its resistance to degradation under ambient or processing conditions, and its mechanical strength. Microcapsules can have the same shape or different shapes, and can have the same size or different sizes. In some instances, the microcapsules can be substantially spheroidal or spherical, and can have sizes ranging from about 0.01 to about 7,000 microns, such as from about 0.1 to about 4,000 microns, from about 0.1 to about 100 microns, from about 0.1 to about 500 microns, or from about 0.5 to about 50 microns. Thus, for example, the microcapsules can have sizes ranging from about 15 to about 25 microns.

As used herein, the term “set” refers to a collection of one or more elements. Thus, for example, a set of microcapsules can include multiple types of microcapsules.

As used herein, the terms “integral” and “integrally” refer to a non-discrete portion of an object. An integrally formed portion of an object can differ from one that is coupled to the object, since the integrally formed portion of the object typically does not form an interface with a remaining portion of the object.

As used herein, the term “latent heat” refers to an amount of heat absorbed or released by a substance (or a mixture of substances) as it undergoes a transition between two states. Thus, for example, a latent heat can refer to an amount of heat that is absorbed or released as a substance (or a mixture of substances) undergoes a transition between a liquid state and a solid state, a liquid state and a gaseous state, a solid state and a gaseous state, or two solid states.

As used herein, the term “transition temperature” refers to a temperature at which a substance (or a mixture of substances) undergoes a transition between two states. Thus, for example, a transition temperature can refer to a temperature at which a substance (or a mixture of substances) undergoes a transition between a liquid state and a solid state, a liquid state and a gaseous state, a solid state and a gaseous state, or two solid states.

As used herein, the term “phase change material” refers to a substance (or a mixture of substances) that has the capability of absorbing or releasing heat to reduce or eliminate heat transfer at or within a temperature stabilizing range, and which is reusable as a phase change material. For instance, the phase change material in the context of the present invention does not evaporate or otherwise become diminished in quantity due to the act of changing phase. This is in contrast to an “evaporative” material, which by changing phase from a liquid to a gas, cannot be reused in the application of the present invention. A temperature stabilizing range can include a specific transition temperature or a range of transition temperatures. In some instances, a phase change material can be capable of inhibiting heat transfer during a period of time when the phase change material is absorbing or releasing heat, typically as the phase change material undergoes a transition between two states. This action is typically transient and will occur until a latent heat of the phase change material is absorbed or released during a heating or cooling process. Heat can be stored or removed from a phase change material, and the phase change material of the present invention typically can be effectively recharged by a source of cold. For certain implementations, a phase change material can be a solid/liquid or solid/solid phase change material. A solid/solid phase change material is a type of phase change material that typically undergoes a transition between two solid states, such as via a crystalline or mesocrystalline phase transformation, and hence typically does not become a liquid during use. For certain implementations, a phase change material can be a mixture of two or more substances. By selecting two or more different substances and forming a mixture, a temperature stabilizing range can be adjusted for any desired application. The resulting mixture can exhibit two or more different transition temperatures or a single modified transition temperature when incorporated in a label described herein.

Examples of phase change materials include a variety of organic and inorganic substances, such as hydrocarbons (e.g., straight chain alkanes or paraffinic hydrocarbons, branched-chain alkanes, unsaturated hydrocarbons, halogenated hydrocarbons, and alicyclic hydrocarbons), hydrated salts (e.g., calcium chloride hexahydrate, calcium bromide hexahydrate, magnesium nitrate hexahydrate, lithium nitrate trihydrate, potassium fluoride tetrahydrate, ammoriium alum, magnesium chloride hexahydrate, sodium carbonate decahydrate, disodium phosphate dodecahydrate, sodium sulfate decahydrate, and sodium acetate trihydrate), waxes, oils, water, fatty acids, fatty acid esters, dibasic acids, dibasic esters, i-halides, primary alcohols, aromatic compounds, clathrates, semi-clathrates, gas clathrates, anhydrides (e.g., stearic anhydride), ethylene carbonate, polyhydric alcohols (e.g., 2,2-dimethyl-1,3-propanediol, 2-hydroxymethyl-2-methyl-1,3-propanediol, ethylene glycol, pentaerythritol, dipentaerythritol, pentaglycerine, tetramethylol ethane, neopentyl glycol, tetramethylol propane, 2-amino-2-methyl-1,3-propanediol, monoaminopentaerythritol, diaminopentaerytbritol, and tris(hydroxymethyl)acetic acid), metals, and mixtures thereof.

As used herein, the term “polymer” refers to a substance (or a mixture of substances) that includes a set of macromolecules. Macromolecules included in a polymer can be the same or can differ from one another in some fashion. A macromolecule can have any of a variety of skeletal structures, and can include one or more types of monomer units. In particular, a macromolecule can have a skeletal structure that is linear or non-linear. Examples of non-linear skeletal structures include branched skeletal structures, such those that are star branched; comb branched, or dendritic branched, and network skeletal structures. A macromolecule included in a homopolymer typically includes one type of monomer unit, while a macromolecule included in a copolymer typically includes two or more types of monomer units. Examples of copolymers include statistical copolymers, random copolymers, alternating copolymers, periodic copolymers, block copolymers, radial copolymers, and graft copolymers. In some instances, a reactivity and a functionality of a polymer can be altered by addition of a functional group such as an amine, an amide, a carboxyl, a hydroxyl, an ester, an ether, an epoxide, an anhydride, an isocyanate, a silane, a ketone, an aldehyde, or an unsaturated group. Also, a polymer can be capable of cross-linking, entanglement, or hydrogen bonding in order to increase its mechanical strength or its resistance to degradation under ambient or processing conditions.

Examples of polymers include polyamides, polyamines, polyimides, polyacrylics (e.g., polyacrylamide, polyacrylonitrile, and esters of methacrylic acid and acrylic acid), polycarbonates (e.g., polybisphenol A carbonate and polypropylene carbonate), polydienes (e.g., polybutadiene, polyisoprene, and polynorbornene), polyepoxides, polyesters (e.g., polycaprolactone, polyethylene adipate, polybutylene adipate, polypropylene succinate, polyesters based on terephthalic acid, and polyesters based on phthalic acid), polyethers (e.g., polyethylene glycol or polyethylene oxide, polybutylene glycol, polypropylene oxide, polyoxymethylene or paraformaldehyde, polytetramethylene ether or polytetrahydrofuran, and polyepichllorohydrin), polyfluorocarbons, formaldehyde polymers (e.g., urea-formaldehyde, melamine-formaldehyde, and phenol formaldehyde), natural polymers (e.g., cellulosics, chitosans, lignins, and waxes), polyolefins (e.g., polyethylene, polypropylene, polybutylene, polybutene, and polyoctene), polyphenylenes, silicon containing polymers (e.g., polydimethyl siloxane and polycarbomethyl silane), polyurethanes, polyvinyls (e.g., polyvinyl butyral, polyvinyl alcohol, esters and ethers of polyvinyl alcohol, polyvinyl acetate, polystyrene, polymethylstyrene, polyvinyl chloride, polyvinyl pryrrolidone, polymethyl vinyl ether, polyethyl vinyl ether, and polyvinyl methyl ketone), polyacetals, polyarylates, alkyd based polymers (e.g., polymers based on glyceride oil), copolymers (e.g., polyethylene-co-vinyl acetate and polyethylene-co-acrylic acid), and mixtures thereof.

The term “encapsulated phase change material” refers to microcapsules of phase change material, and is abbreviated as EPCM.

### Selection of Phase Change Materials

A transition temperature of the phase change material typically correlates with a desired temperature or a desired range of temperatures that can be maintained by the phase change material. Regardless of which embodiment is used to create the system 10 of the present invention, it is desirable that article 11 include a phase change material having a transition temperature starting within a range of about negative 17.8 C (−17.8 C) to about 37 C. If it is possible to refrigerate or otherwise cool the phase change article prior to use, it may be desirable to include one or more phase change materials having transition temperatures at the lower end of the range, such as between about negative 17.8 C to about 16 C. If it is impractical to cool the article 11 prior to use, then it may be desirable to only include one or more phase change materials having transition temperatures at the higher end of the range, perhaps about 16 C to about 37 C, or about 23 C to about 37 C, or about 32 C to about 37 C. The selection of the phase change material can be dependent upon other considerations, such as its reactivity or lack of reactivity with a material forming the microcapsules and its resistance to degradation under ambient or processing conditions.

For certain implementations, the phase change material may include a paraffinic hydrocarbon having n carbon atoms, namely a C, paraffinic hydrocarbon such as Standard or Custom MicroPCM Products™ obtained from Microtek Laboratories, Inc., Dayton, Ohio. For example, n-Eicosane has a transition temperature (melting point) of about 37° C. By comparison, n-Octadecane has a transition temperature of about 28° C., n-Hexadecane has a transition temperature of about 18° C., and n-Tetradecane has a transition temperature of about 6° C.

Any number of the n-paraffins noted above (among others not mentioned) may be blended on a micro or macro scale to achieve custom transition temperatures. In one non-limiting embodiment of the present invention, a blend of n-Eicosane and n-Octadecane is created and then encapsulated into microcapsules. In another non-limiting embodiment of the present invention, the n-Eicosane and n-Octadecane are separately encapsulated into microcapsules, and used in an article as a set. Either EPCM may be used to create articles such as those described herein.

### Phase Change Articles

The physical forms which phase change articles of the present invention may take include packaging and other objects such as packets, figurines, tokens, jewelry, or any object that can be formed with an effective amount of a phase change material.

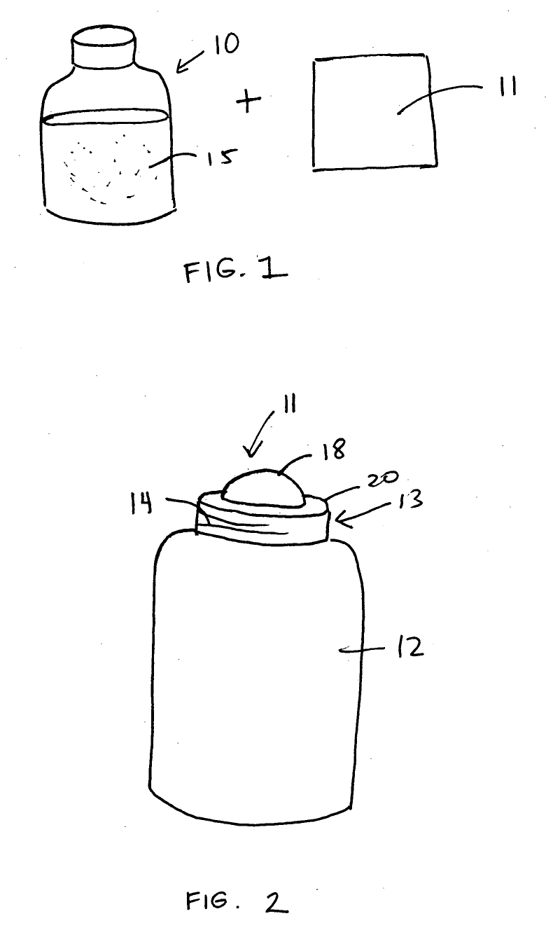

In a first exemplary embodiment of the article 11 of system 10, shown in FIG. 2, is a vessel 12 that incorporates an encapsulated phase change material into its structure. The vessel 12 is a container that may be capable of at least storing and possibly dispensing any type of liquid cooling composition containing a phase change material, a neurosensory material and/or an evaporative material. The vessel 12 may dispense the cooling composition by pouring, or the vessel 12 may dispense through an applicator such as a roller, spray device, or brush. In the alternative, the vessel 12 may be used as an outer package, and be used to contain a non-liquid object. For instance, the vessel 12 may contain packets or bottles of a cooling composition.

Suitable materials for a vessel 12 include any material 1) that may be formed by carving, lathing, molding, thermo molding, blow molding, and the like and 2) that contains effective amounts of a phase change material, such as an EPCM. In one embodiment, the vessel 12 material is a plastic resin blended with an EPCM. One specific example may be a plastic resin blended with about 25 to about 70% by weight EPCM such as paraffin having a transition temperature of about 6 C to about 40 C, to create an EPCM composite material. In another embodiment, the plastic resin may be blended with about 40 to about 60% by weight EPCM.

Suitable plastic resins include high density polyethylene (HDPE), low density polyethylene (LDPE), polyethylene terephthalate (PET), polyvinyl chloride (PVC), polypropylene (PP), polystyrene (PS), and any other resin that may be combined with the encapsulated PCM. The resin may be selected on the basis of its ability to conduct heat transfer. The plastic resins may by made into vessels via injection molding, extrusion, blow molding, or other methods of forming plastic vessels as known in the art. If desired, the EPCM composite material may be a layer formed onto another plastic vessel. The composite containing the EPCM is oriented on an outer surface so that it may be contacted directly by touching the container.

In yet another embodiment, vessel 12 is constructed from a composite of pulverized stone, resin, and EPCM materials. In one non-limiting example, a pulverized quartz, EPCM, and resin composite may be formed by a molding process. For example, about 2% by weight resin combined with about 5% to about 70 percent EPCM, and about 28% to about 93% pulverized stone may be blended and placed into a vessel mold. Separate molded pieces may be joined by a liquid impermeable resin or other suitable adhesive. Suitable stone includes quartz, granite, and the like.

Vessels 12 may be combined with an applicator 13 such as a spray head, a roll-on device, a nozzle, or a brush device as are known in the art. For example, a vessel may contain a liquid neurosensory formulation containing icilin, menthol, or the like; an evaporative formulation containing water, alcohol, or the like; and/or a phase change material. Referring to FIG. 2, the vessel 12 may include an opening surrounded by a threaded collar 14. A roll-on device which includes a roller ball 18 and socket member 20 may be selectively attached to the threaded collar 14. The roller ball is desirably glass, metallic, or any material having a coefficient of conductivity such that it feels cools to the touch via conduction. Roller ball designs are known in the art, such as those disclosed in U.S. Pat. Nos. 2,749,566; 2,883,690; 2,981,968; 2,997,731; 3,048,881; 4,021,125; 4,168,128; and 4,326,508, incorporated herein to the extent that they are consistent with the present invention. Of course, the applicators may connect to a vessel 12 in other ways, such as by clamping, an interference fit, or the like. In the alternative, applicators may be permanently attached to the vessel with an adhesive.

Vessel 12 may also be coated with a phase change material either in addition to the embodiments described above, or as the sole source of phase change material in the vessel 12 structure. The coating material may be applied first to a label-type substrate and then attached to the vessel structure 12, or the coating material may be applied directly onto the vessel 12, with the vessel serving as a substrate, such as the substrate 34 described below.

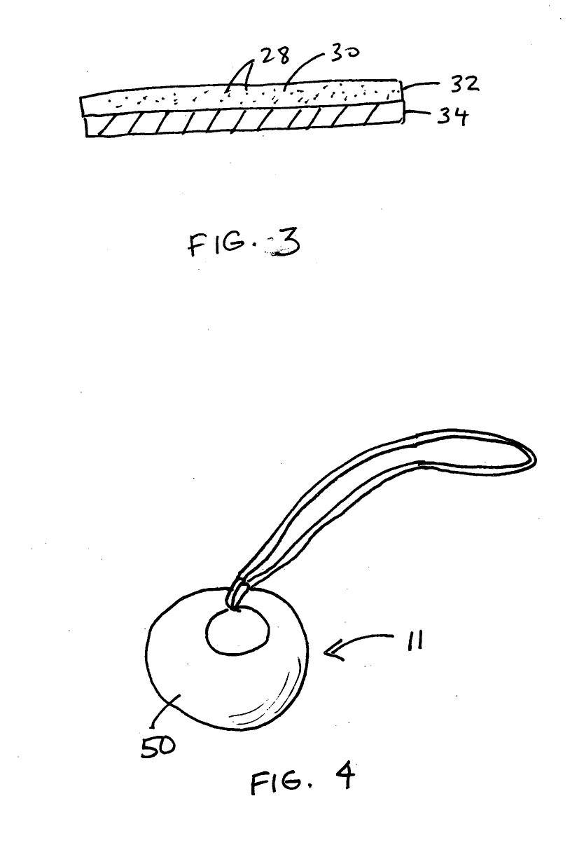

Referring to FIG. 3, the coating material may be made from a suspension of EPCM in a carrier. For example, the carrier may be an ink or paint that once applied to a surface and allowed to dry, forms a layer 32. In a specific example, a substrate 34 is a structural support layer for the coating material, and may be formed of any suitable material, such as a fibrous material, mineral, polymer, metal, or composite thereof. In one particular non-limiting example, the substrate 34 can be formed of sheet material made from a paper, polyester, and polyolefin (e.g. polyethylene or polypropylene, or a polyvinyl). In the alternative, the substrate 34 may be an object such as the vessel 12 described herein, a figurine, or other items that are not in sheet form, described below. The selection of a material forming the substrate 34 can be dependent upon certain considerations, such as its ability to facilitate formation of a layer 32, its flexibility, its resistance to degradation under ambient or processing conditions, and its mechanical strength.

The coating layer 32 can be applied to substrate 34 using roll coating, such as direct gravure coating, reverse gravure coating, differential offset gravure coating, or reverse roll coating; screen coating; spray coating, such as air atomized spraying, airless atomized spraying, or electrostatic spraying; extrusion coating; or transfer coating. After the coating layer is applied to the surface of substrate 34, the blend can be cured, dried, cross-linked, reacted, or solidified to form the layer 32.

In the illustrated embodiment of FIG. 3, the layer 32 is formed of a binder 30 and a set of microcapsules 28 that are dispersed in the binder 30. The binder 30 can be any suitable material that serves as a matrix within which the microcapsules are dispersed, and that couples the microcapsules 28 to the substrate 34. The binder 30 can provide other desired functionality, such as offering a degree of protection to the microcapsules 28 against ambient or processing conditions or against abrasion or wear during use. For example, the binder 30 can be a polymer or an ink medium used in certain printing techniques. The selection of the binder 30 can be dependent upon other considerations, such as based on its affinity for the microcapsules 28, its ability to reduce or eliminate heat transfer, its flexibility, its coating-forming ability, its resistance to degradation under ambient or processing conditions, and its mechanical strength. Thus, for example, the binder 30 can be selected based on its affinity for the microcapsules 28 so as to facilitate dispersion of the microcapsules within the binder 30. Such affinity can be dependent upon, for example, similarity in polarities, hydrophobic characteristics, or hydrophilic characteristics of the binder 30 and a material forming the microcapsules 28. For example, the binder 30 can be selected to be the same as or similar to a material forming the microcapsules 28. Advantageously, such affinity can facilitate incorporation of a higher loading level as well as a more uniform distribution of the microcapsules 28 within the layer 34.

As mentioned, articles 11 may take the form of figurines, tokens, jewelry, packets or any object that can be formed with and/or coated with an effective amount of a phase change material such as an EPCM. In one embodiment of the present invention, an article 11 takes the form of jewelry at least partially made from a phase change material. For example, beads or pendants made with a phase change material may be included in the system 10. In one non-limiting example shown in FIG. 4, a decorative pendant 50 may include a metallic skin (e.g. silver), covering a core of suitable phase change material (e.g. paraffin). The metallic skin may be an electroplated layer covering at least one surface of a carrier metal such as brass or the like, or a homogeneous layer of a single material. The paraffin may be formulated to have a single or multiple transition temperatures, as described above. In one embodiment, the pendant 50 may have a mass suitable to maintain the transition temperature for about 5 minutes or more. It is further contemplated that the pendant 50 may be made from a composite material such as pulverized stone, resin, and EPCM as described above with respect to vessel 12. It is even further contemplated the pendant may have a core member of plastic, wood, or the like, and be coated with a material layer 32, as described above.

Instead of a pendant form, the article 11 could take the form of a figurine or a token such as a ball or coin. Such articles may be constructed from the same materials as described for the vessel 12 or pendant 50. In one non-limiting example shown in FIG. 6, there is a token 80 that is made from a molded composite made with a resin and EPCM, or any other materials as described previously in relation to the jewelry 50 and vessel 12.

The token 80 may be plain, or bear indicia. The indicia may be a graphic such as a symbol, flower, person, object, animal, or the like. It is further contemplated that the graphic may at least in part, be an inspirational message.

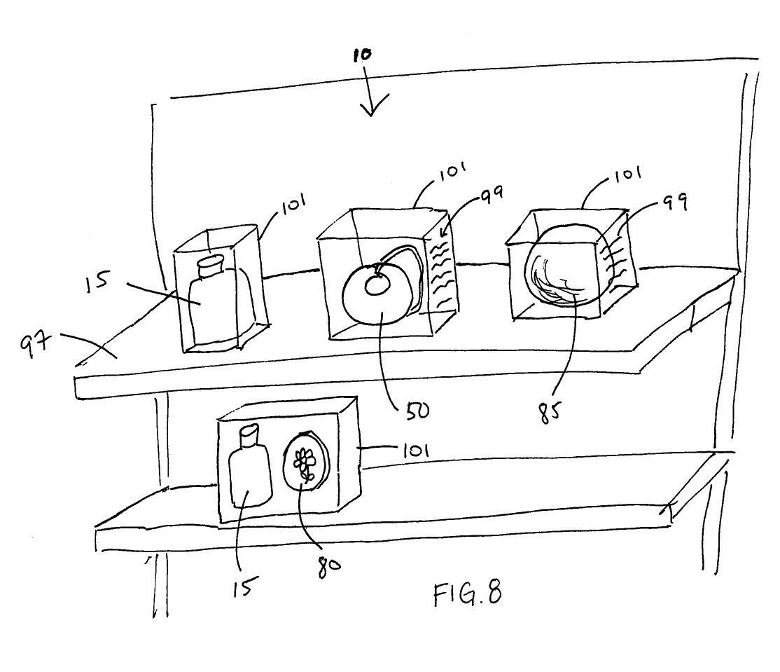

Instead of a token shape, the article 11 may be a figurine 85 as simple as the sphere shown in FIG. 7, or a more complex shape. It is desirable to select a shape that allows as much area to contact the user's skin as possible. Very complex shapes may not conform to the skin, and without direct contact with the users skin, thermal conductivity is hindered. A ball may be desirable as an article intended to be held in the hand. The figurine 85 may be made from the same materials as described herein for the jewelry 50 and vessel 12.

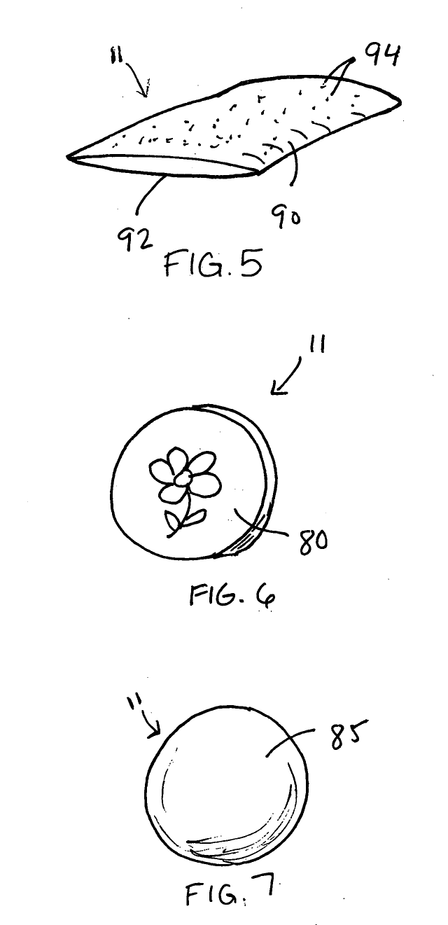

In yet another embodiment of the present invention as shown in FIG. 5, article 11 takes the form of a packet 90 containing EPCM 94, similar to a bean bag. The packet 90 is an envelope 92 filled with a quantity of EPCM 94. The envelope is a flexible material, desirably a material that is able to conduct heat from the user's body to the EPCM 94. For example, a 2-3 mil thick sheet of polyethylene may be formed into an envelope 92 for containing EPCM having a size of about 4000 to about 7000 microns. The sheet may include an array of apertures such that the EPCM does not leak from the envelope, but allows skin contact with a portion of the EPCM. Desirably, the EPCM 94 are shaped so that they may pack tightly against one another for purposes of thermal conduction. Providing that the envelope is not tightly packed with EPCM, a user may mold the packet 90 against any part of the body.

Very simply, envelope 92 may be constructed by aligning together the edges of two identically shaped sheets and bonding the edges together. Envelope 92 may be made from rectilinear sheets, or sheets of any geometric shape such as an oval, circle, triangle, and the like. The overall shape of envelope 92 may be fanciful, such a butterfly, flower, heart, or the like. It is contemplated that envelope 92 may be constructed from a single sheet, folded and sealed to form an interior volume. It is further contemplated that envelope 92 may be constructed from more than two sheets. For instance, as seen in FIG. 5, envelope 92 may have a top sheet 93, an opposite bottom sheet (not shown), and a pair of side panels 95 (only one side shown).

In another embodiment of the packet 90, the envelope 92 is a liquid impermeable layer of material such as polyethylene or nonwoven, and the phase change material is provided as a single mass. For example, a bar of paraffin or the like may be enclosed within the envelope. The bar may be initially shaped to fit against a select portion of the body, such as the lumbar region of the back. Packet 90 may also take the form of a pouch, pillow, or the like (not shown).

System 10 may be packaged together as a kit. In one embodiment, the system 10 includes a cooling composition contained within a vessel 12 serving as the article 11. If desired, a second article 11 may be included in the kit.

In another embodiment, the system 10 includes a cooling composition contained in a package that is not constructed with a phase change material, and a first article 11, taking the form of a vessel, a packet, a figurine, jewelry, a token, and the like. The kit may further include a second article 11, having the same or different form as the first article 11.

Any of the articles 11 of the present invention may include a fragrance, and in particular an essential oil for an aromatherapy effect.

Referring to FIG. 8, a method of marketing the skin cooling system 10 generally includes the steps of (a) providing a skin cooling composition, (b) providing an article comprising a phase change material, and (c) providing instructions on how to use the skin cooling composition and the article to provide a cooling sensation on human skin. For example, the article 11 and the cooling composition 15 may be offered to shoppers, users, or choosers by catalog, web site, written or displayed advertising, in a retail store (such as on a shelf or kiosk), or by a salesperson. The article 11 and the cooling composition may be physically separated by a distance, for instance an cooling composition 15 in a bottle, and an article 11 may be displayed side-by-side on a retail store shelf 97. However, it is contemplated that the cooling composition 15 may be contained within a vessel-type article 11. Regardless, a set of instructions 99, either written on paper or available via an electronic medium such as a web site, are included with one or both of the article 11 and the cooling composition 15. For instance, any housing 101 used to contain the article 11, cooling composition, or a kit containing both may further house an instruction pamphlet (not shown) or have the instructions written directly thereon. The instructions 99 may describe how a synergistic benefit can be obtained by using both the article 11 and the cooling composition 15 to achieve a skin cooling sensation.

Although only a few exemplary embodiments of this invention have been described in detail above, those skilled in the art will readily appreciate that many modifications are possible in the exemplary embodiments without materially departing from the novel teachings and advantages of this invention.

1. A cooling system comprising:
a human skin cooling composition comprised of a neurosensory component; and
an article comprising a first phase change material,
wherein the human skin cooling composition and the article are secured together by a package to form a kit.

2. The cooling system of claim 1 wherein the article comprises a second phase change material having a different transition temperature than the first phase change material.

3. The cooling system of claim 2 wherein the article comprises a third phase change material having a different transition temperature than both the first phase change material and the second phase change material.

4. The cooling system of claim 1 wherein the article comprises a vessel.

5. The cooling system of claim 4 wherein the first phase change material comprises a plurality of microcapsules.

6. The cooling system of claim 5, the article comprises a second phase change material that has a transition temperature that is different than that of the first phase change material.

7. The cooling system of claim 4 wherein the article vessel comprises an applicator.

8. The cooling system of claim 1 wherein the article comprises a token or a figurine.

9. The cooling system of claim 8 wherein the first phase change material comprises a plurality of microcapsules.

10. The cooling system of claim 1 wherein the article comprises a packet.

11. The cooling system of claim 10 wherein the article comprises a first phase change material in the form of a single mass.

12. The cooling system of claim 1 wherein the first phase change material comprises a plurality of microcapsules.

13. The cooling system of claim 12 wherein the plurality of microcapsules and a binder together comprise a coating layer.

14. The cooling system of claim 1 wherein the article comprises a fragrance.

15. The cooling system of claim 1 further comprising a bonus article comprising a second phase change material.

16. The cooling system of claim 15 wherein the second phase change material is different from the first change material.

17. A cooling system comprising:
a human skin cooling composition comprised of a neurosensory component; and
a vessel containing the cooling composition, the vessel comprising a plurality of microcapsules.

18. The cooling system of claim 17 wherein the plurality of microcapsules and a binder together comprise a coating layer.

19. The cooling system of claim 17 wherein the vessel comprises a set of microcapsules.

20. A method of marketing a skin cooling system, the method comprising the steps of:
providing a skin cooling composition;
providing an article comprising a phase change material; and
providing instructions on how to use the skin cooling composition with the article to provide a cooling sensation on human skin.
